(19) United States
(12) Patent Application Publication
(10) Pub. No.: US 2008/0015510 A1
(43) Pub. Date: Jan. 17, 2008

(54) Ampoule Recognition

(76) Inventors: Pascal Sandoz, Prangins (CH); Beat Steffen, Saanen (CH); Markus Tschirren, Kirchberg (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/461,896

(22) Filed: Aug. 2, 2006

Related U.S. Application Data
(63) Continuation of application No. PCT/CH05/00041, filed on Jan. 26, 2005.

(30) Foreign Application Priority Data
Feb. 3, 2004 (DE) 10 2004 005 311.1

(51) Int. Cl. A61M 5/00

(52) U.S. Cl. 604/187

(57) ABSTRACT

An injection device for dispensing a medical substance in doses, comprising a connecting point at which an ampoule can be inserted into the device, wherein in the area of the connecting point, at least one transfer element is provided which can engage with a surface structure of an ampoule and/or can be moved to transfer surface structure information to a sensor. An ampoule for accommodating a medical substance, comprising a surface structure which comprises at least one recess and/or protrusion in the circumferential direction is encompassed.

### CROSS-REFERENCE TO RELATED APPLICATION(S)

This application is a continuation of International Patent Application No. PCT/CH2005/000041, filed Jan. 26, 2005, which claims priority to German Application No. DE 10 2004 005 311.1, filed on Feb. 3, 2004, the contents of which are incorporated in their entirety by reference herein.

### BACKGROUND

The present invention relates to devices for administering, injecting, delivering or dispensing substances, and to methods of making and using such devices. More particularly, it relates to an ampoule recognition system for administering devices such as an injection apparatus or devices, including injection pens, into which an ampoule containing a fluid substance or product can be inserted.

Devices for administering substances, e.g., medicinal or therapeutic substances or products, from ampoules are known in the form of injection devices, including portable injection apparatus or pens. Such devices are used, for example, in insulin treatment, wherein ampoules filled with the substance to be administered are coupled to an administering device—often referred to as a pen or an injection pen—to dispense the substance contained in the ampoule to a patient via the administering device. A multitude of substances are administered in this way, for example preparations comprising insulin for diabetes, growth hormones (hGH—human growth hormone) for disturbed growth, erythropoietin (Epo) for renal insufficiency or general erythrocytopenia, α-interferon for example for hepatitis or cancer treatment or potentiating agents. Thus, the often geometrically identical ampoules are often filled with different substances or also different concentrations of substances to be administered.

To reduce the danger of mixing up containers or ampoules containing different substances, differently embodied administering devices are known, wherein respectively corresponding containers can be received in a certain administering device.

WO 98/00187 discloses a container with a color coding which can be attached to it, consisting of a number of different colored fields, wherein a property of a container or its contents can be recognised by an optical sensor system.

WO 01/56635, belonging to the owner of the present application, discloses a container and a device for administering a substance, wherein a recognition element is assigned to the container.

DE 101 47 973 A1, belonging to the owner of the present application, discloses an administering device which is coupled to an ampoule and comprises at least two Hall sensors which can be arranged at preset positions to recognise the presence or absence of at least two permanent magnets which are fixedly connected to an ampoule which can be inserted into the administering device.

FIG. 5 shows an ampoule 1 with rod-shaped magnets 2 which serve as recognition elements. The ampoule 1 is screwed completely into a pen 8, such that the magnets 2 in the ampoule 1 each lie opposite one of four asymmetrically arranged sensors 3 arranged on a circuit board 4 and fixedly connected to the pen 8. To correctly position the circuit board 4, protrusions (not shown) are provided on the pen 8 which engage with lateral cavities in the circuit board 4 and thus position the circuit board 4 in the pen 8 such that it is secured against rotating. To be able to transfer the signals from the sensors 3 to an evaluation logic or chip (not shown), a flexible line connection 5 comprising a number of adjacent lines is connected to the circuit board 4. The pen 8 further comprises a battery (not shown) by which the sensors 3 and the evaluation logic can be supplied with power. Above a mechanism holder, in which a threaded rod is guided which can pass through the circuit board 4 at a central opening 4a, is a display device on which the ampoule type or ampoule contents detected by the magnets 2 and sensors 3 can be displayed. A spring between the ampoule 1 and the circuit board 4 ensures a stable fit for the ampoule 1.

### SUMMARY

An object of the present invention to provide an administering device, and a method of using it, which enables costs to be reduced when repeatedly using the device with different ampoules. Devices in accordance with the present invention include injection devices, including such devices known as injection pens.

In one embodiment, the present invention comprises an injection device for dispensing a medical substance in doses, comprising a connecting point at which an ampoule can be inserted into the device, wherein in the area of the connecting point, at least one transfer element is provided which can engage with a surface structure of an ampoule and/or can be moved to transfer surface structure information to a sensor. An ampoule for accommodating a medical substance, comprising a surface structure which comprises at least one recess and/or protrusion in the circumferential direction, is encompassed.

In one embodiment, the present invention comprises a system comprising an ampoule for accommodating a medicinal substance, comprising a surface structure on an ampoule surface, said surface structure comprising at least one of a recess or a protrusion extending in a circumferential direction at least partially around the ampoule, and an administering device for dispensing a medicinal substance in doses, comprising a connecting point at which the ampoule can be inserted into the device and at least one transfer element adjacent to the connecting point, the at least one transfer element either engageable with the surface structure of the ampoule or moveable by the surface structure to transfer surface structure information to a sensor operably associated with the administering device.

In accordance with one embodiment of the present invention, an administering device such as an injection pen for dispensing a medical substance in doses from an ampoule which can be coupled to the pen comprises a connecting point at which the ampoule can be attached or inserted, wherein in the area of the connecting point, at least one transfer element is provided which can engage with a surface structure of an ampoule or interact with a surface or the ampoule itself, such that the at least one transfer element can be moved, for example shifted and/or tilted, to transfer information, yielded by the surface or surface structure or the ampoule, to at least one sensor inserted into the pen or coupled to the pen. It is thus possible to recognize from the surface structure or a profile of the ampoule itself what type of ampoule is present and/or what substance is contained in a particular ampoule. It is no longer necessary to integrate magnets into the ampoule, as for example proposed in DE 101 47 973 A1, but rather magnets or other signal-emitting active or passive elements can be attached in the pen, for example to the transfer elements, whereby only one set of magnets is needed for the pen as a non-disposable component or part thereof, and the ampoules—often embodied as disposable—do not have to be individually provided with a new set of magnets.

In some embodiments, the magnets associated with or attached to or in the pen are moved, for example rotated and/or shifted, relative to the at least one sensor associated with or attached to or in the pen, such as for example Hall sensors or Reed contacts, by a mechanical development or feature on the ampoule, for example on the facing surface of the ampoule, which when introduced—for example slid and/or rotated—into the pen, shifts one or more of the magnets relative to respectively assigned sensors, in accordance with the profile on the surface of the ampoule, such that it is possible to ascertain from the sensor signals what profile the ampoule has. If particular ampoule contents are assigned to a particular ampoule profile, then it is possible to deduce from the sensor signals what ampoule type has been inserted into the pen.

In some preferred embodiments, the connecting point provided on the pen has a guide, comprising for example an inner thread, an outer thread, grooves and/or protrusions, for inserting or attaching an ampoule, such that the ampoule can be inserted, for example slid or screwed, into the pen in a defined way.

In some embodiments, a limiting element is provided at one end of the guide, for example a stopper, a latching mechanism, a spring biased latch, or an interruption of a thread provided in the pen, such that the ampoule can be introduced, received or inserted into the pen up to a defined position.

In some embodiments, the at least one transfer element provided in the pen or connected to the pen can be moved, for example pivoted and/or shifted, relative to the pen, wherein a pivoting transfer element is arranged on the pen such that the pivot axis of the transfer element is approximately perpendicular on the centre axis of an ampoule to be inserted. If a shifting transfer element is provided, then it is advantageous if the transfer element can be shifted approximately parallel to the center axis of the ampoule to be introduced, such that, for example, different transfer elements can be slid into the pen to different extents, in accordance with different recesses on the surface of the ampoule.

In some preferred embodiments, at least one permanent magnet is fixedly connected to the at least one transfer element, wherein the permanent magnet or magnets is or are arranged on the transfer elements such that when an ampoule is introduced, they are moved either toward or away from assigned Hall sensors or Reed switches in accordance with a surface profile of the ampoule.

In some embodiments, at least one of the transfer elements or each of the transfer elements is biased or urged to move in a selected motion, for example by a spring, such that a transfer element is ejected from an initial position by an inserted or screwed-in ampoule, against the biasing force of the spring, and—after the ampoule is removed—is automatically moved, for example pivoted or slid, back to the initial position by the spring force.

In some embodiments, one or more sensors, such as magnetic field sensors, Hall sensors, etc., are arranged in the pen and are assigned to magnets on transfer elements to detect whether a transfer element has been moved or not.

In accordance with another aspect of the present invention, the invention relates to an ampoule having a surface profile which enables the ampoule and therefore the contents of the ampoule to be clearly identified by an injection device or injection pen as described above. In some embodiments, the surface profile of the ampoule is on the side or surface of the ampoule introduced or inserted into the pen first (which side or surface may be referred to as the facing side), and comprises one or more recesses in the circumferential direction on the facing side, with which transfer elements of the pen can engage, and one or more protrusions which transfer elements of the pen can slide or push away. The present invention further comprises a set of such ampoules which exhibit different surface profiles, wherein one ampoule can be distinguished from another. The present invention further comprises to a system and method comprising an injection device or pen as described above and an ampoule as described above.

### BRIEF DESCRIPTION OF THE DRAWINGS

FIG. 1 is an exploded view of an embodiment of an ampoule accommodating and recognizing device which can be used with and/or inserted into an injection device;

FIGS. 2A-2F depict switched positions of transfer elements at different rotational angles of an ampoule screwed into an injection device or injection pen;

FIGS. 3A and 3B are views of a circuit board with sensors attached to it;

FIGS. 4A and 4B are schematic illustrations of the functional principle of magnetic detection; and

FIG. 5 is a perspective view of an ampoule inserted into an injection device or injection pen.

### DETAILED DESCRIPTION OF THE DRAWINGS

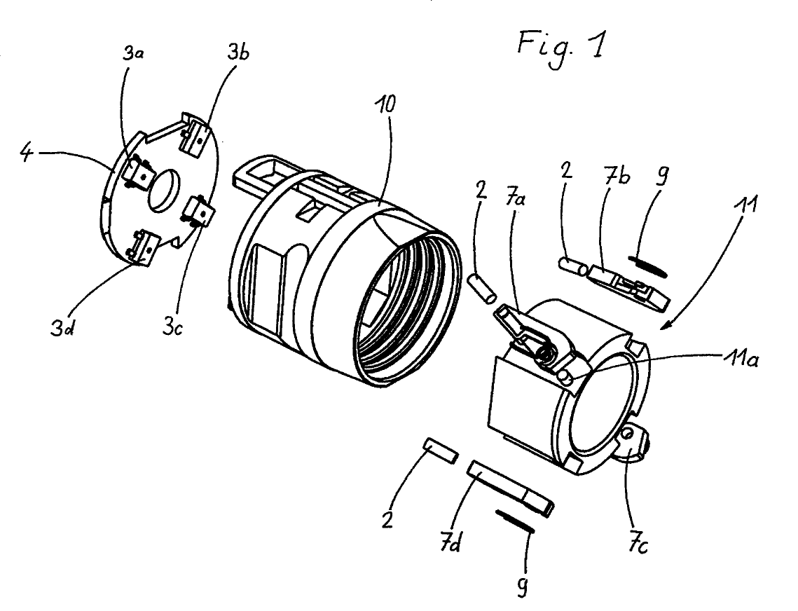

FIG. 1 is an exploded view of an embodiment of an ampoule accommodating and recognizing device in accordance with the present invention, comprising an ampoule accommodating element 10 comprising an inner thread 10a into which an ampoule 1 comprising an outer thread can be screwed, as schematically shown in FIG. 5. A circuit board 4 with four Hall sensors 3a-3d arranged on it is attached to the lower side of the ampoule accommodating device 10 shown on the left in FIG. 1, wherein the four Hall sensors 3a-3d exhibit approximately the same spacing in the circumferential direction of the circuit board 4, but could also be assigned asymmetrically, or in any pattern respective or corresponding to respective or corresponding transfer elements. The transfer element support 11 is inserted into the ampoule accommodating device 10, and four journals 11a protruding radially outwards are provided at approximately the same spacing, onto each of which a transfer element 7 can be placed. Each transfer element 7 comprises a receptacle 7a for plugging in a rod-shaped permanent magnet 2 and a receptacle for a spring 9, such that a transfer element 7 attached to the journal 11a of the transfer element support 11 is pushed by the force of the spring 9 against a protrusion on the outer side of the transfer element support 11, into a position in which a part of the transfer element 7 protrudes beyond an abutting surface 11b of the transfer element support 11 at cavities 11c on the abutting surface 11b provided for this purpose. The transfer element support 11 inserted into the ampoule accommodating device 10 is positioned in the ampoule accommodating device 10 such that the permanent magnet 2 connected to the transfer elements 7 do not lie opposite the Hall sensors 3 in the initial position and are moved to the Hall sensors 3 of the circuit board 4 attached to the ampoule accommodating device 10 when the parts of the transfer elements 7 protruding beyond the abutment 11b of the transfer element support 11 are pushed in against the force of the spring 9, for example by a facing surface of an ampoule 1 screwed into the ampoule accommodating device 10.

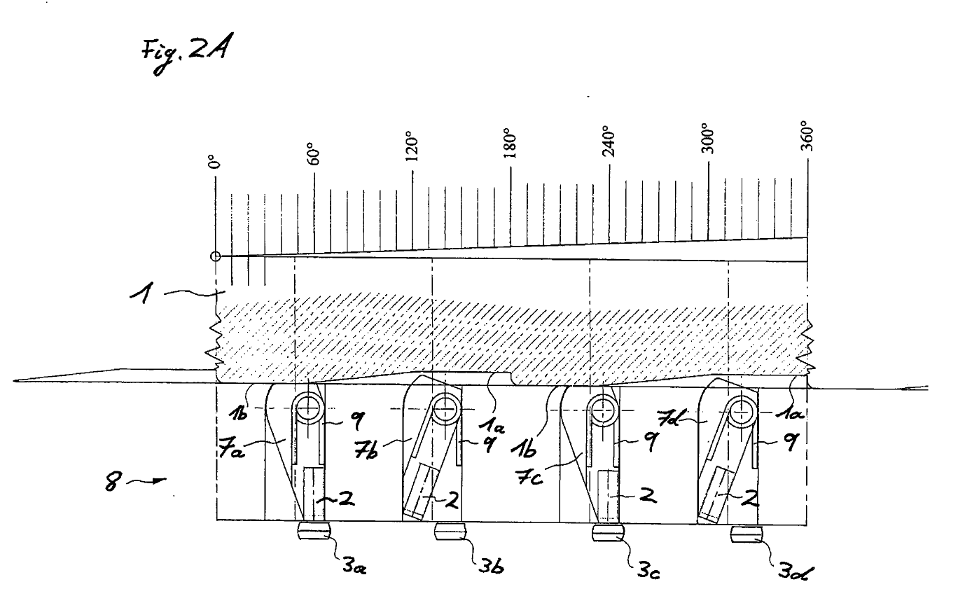

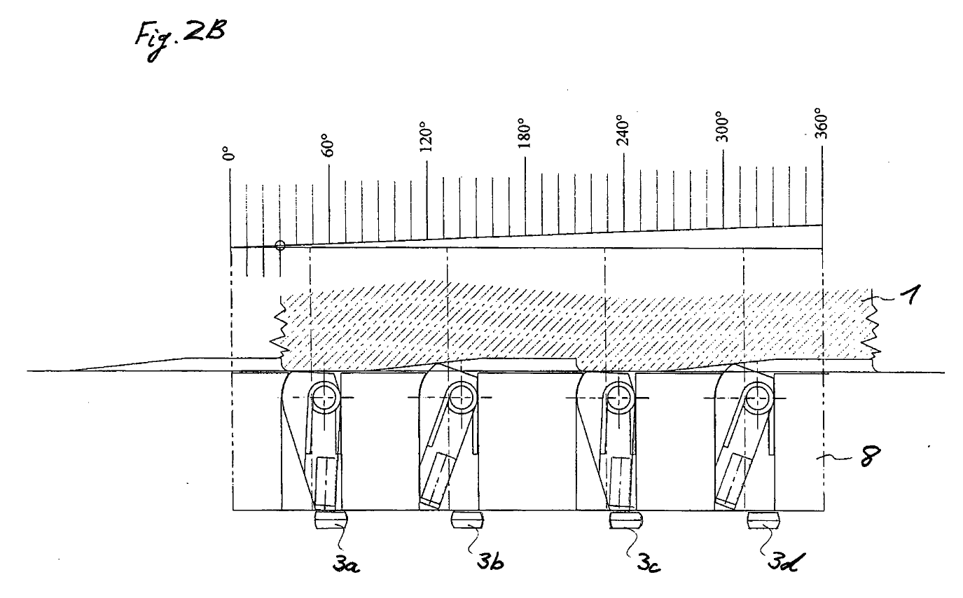

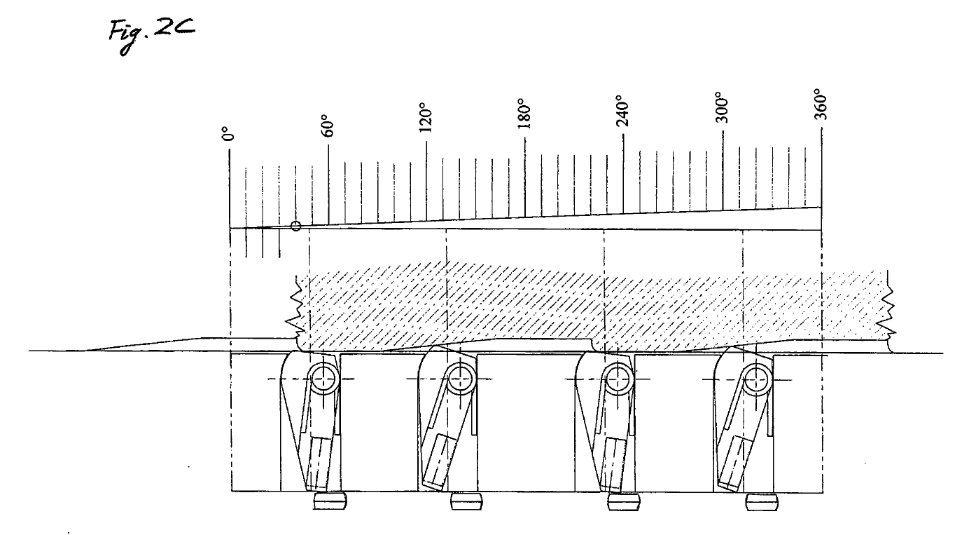

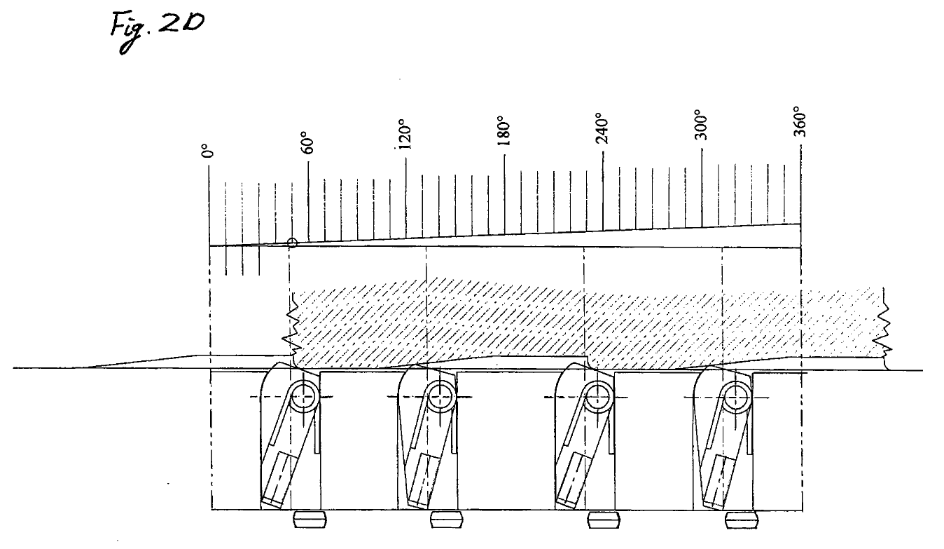

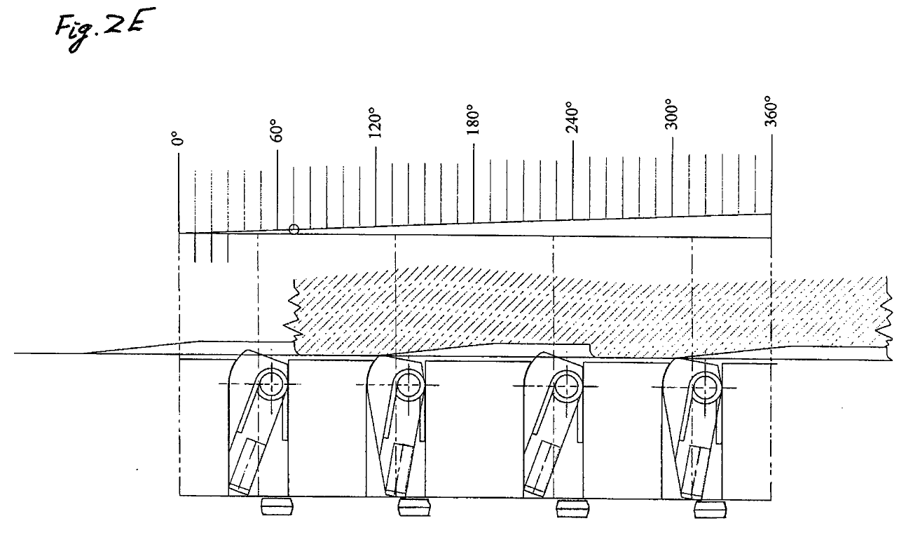

FIGS. 2A-2F show different states of four transfer elements 7a-7d inserted in a pen 8, at different rotational angles of an ampoule 1 screwed into the pen 8. FIG. 2A shows a state when an ampoule 1 is completely screwed into a pen 8, wherein a profile provided on the facing side of the ampoule 1, when it is completely screwed in, comprises recesses 1a at the points of the transfer elements 7b and 7d and protrusions 1b at the points of the transfer elements 7a and 7c, moving the transfer elements 7a and 7c against the force of the spring 9 such that the permanent magnets 2 connected to the transfer elements 7a and 7c are moved to corresponding Hall sensors 3a and 3c of the pen 8. The transfer elements 7b and 7d are held by the force of the spring 9 in a position in which a part of the transfer element 7 is pushed into a recess 1a on the facing surface of the ampoule 1, such that the permanent magnets 2 connected to the transfer elements 7b and 7d do not lie opposite the corresponding Hall sensors 3b and 3d.

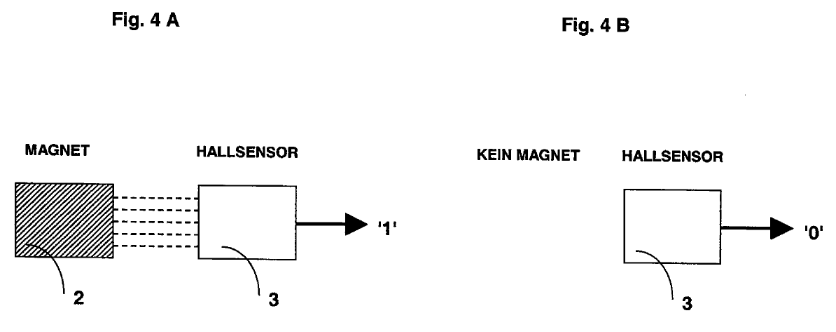

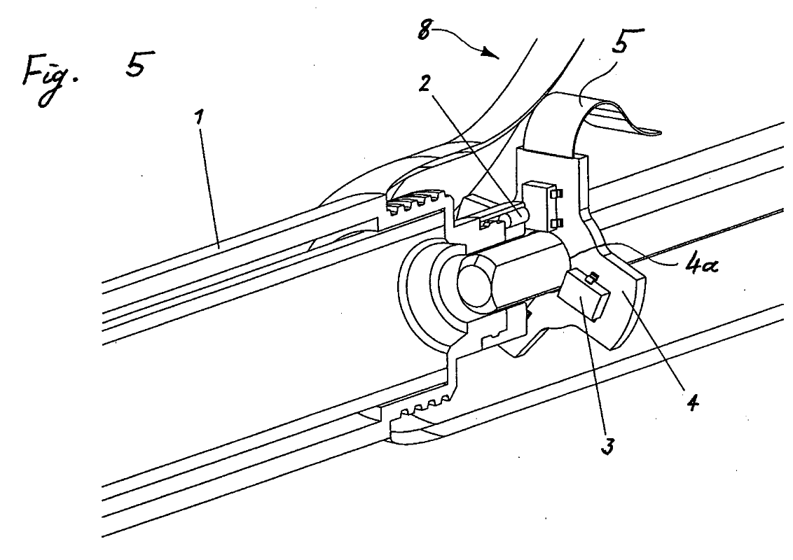

As schematically shown in FIGS. 4A and 4B, a Hall sensor 3 emits a signal which can, for example, be understood as a logical “1”, when a magnet 2 is lying opposite the Hall sensor 3, and outputs another signal which can for example be understood as a logical “0”, when no magnet is lying opposite the Hall sensor 3.

The position of the permanent magnets 2 relative to the Hall sensors 3a-3d shown in FIG. 2A would thus, for example, generate a coding signal “1010” which enables a particular ampoule type to be deduced.

FIGS. 2B-2F show different states of the transfer elements 7a-7d at different rotational angles of the ampoule 1, namely 30°, 40°, 50°, 70° and 150°, wherein it can be seen from FIG. 2B that even at a rotational position of the ampoule 1 which deviates by 30° from one optimum rotational position shown in FIG. 2A, the signal generated by the Hall sensors 3a-3d is “1010”. This enables a rotational tolerance on the ampoule 1 screwed into the pen 8 to measure 30°, without an incorrect signal being generated. At larger rotational angles of 40°, 50° and 70°, as shown in FIGS. 2C-2E, the transfer elements 7a and 7c are moved away from the sensors 3a and 3c.

FIG. 2F shows the position when the ampoule 1 is rotated 150° out of the pen 8, wherein all the transfer elements 7a-7d are in a resting position and all the Hall sensors 3a-3d emit a logical “0” as their signal.

The table below shows the switched state for four Hall sensors a-d in an embodiment of the invention in which the Hall sensors a and c are used to check whether an ampoule is inserted and the Hall sensors b and d are used to recognize a particular ampoule type. The Hall sensors b and d only recognize an ampoule type when the Hall sensors a and c output a logical “1” as their signal. The first line shows the switched state when no ampoule is inserted, wherein all the Hall sensors output a logical “0” as their signal. If an ampoule not having a surface structure in accordance with the invention is inserted, then all the transfer elements 7 of the transfer element support 11 are pushed in, such that all the Hall sensors output a logical “1”. In this case, a signal can, for example, be generated which indicates, for example, that the concentration of the ampoule contents is to be set on the pen by the user, as described in application 103 51 732.4-44 belonging to the owner of this application, the teaching of which with respect to manually setting an ampoule concentration is incorporated into this application.

If an ampoule in accordance with the present invention is inserted into a pen in accordance with the present invention, then it is possible, in accordance with different surface profiles, to recognize what type of ampoule is present through the different switched states of the Hall sensors b and d, wherein—as opposed to an ampoule inserted into the pen in accordance with the present invention but not having a surface structure in accordance with the present invention—at least one of the Hall sensors b and d outputs a logical “0” as its signal, such that an ampoule in accordance with the invention, when completely screwed into the pen, comprises for example protrusions 1b in the area of the sensors a and c and at least one recess 1a in the area of the sensors b and d and can thus be clearly identified by the at least one recess 1a schematically shown in FIG. 2A.

Hall sensor

| | a | b | c | d |
|---|---|---|---|---|
| no ampoule | 0 | 0 | 0 | 0 |
| ampoule not having | 1 | 1 | 1 | 1 |
| surface structure | | | | |
| ampoule No. 1 | 1 | 1 | 1 | 0 |
| ampoule No. 2 | 1 | 0 | 1 | 1 |
| ampoule No. 3 | 1 | 0 | 1 | 0 |

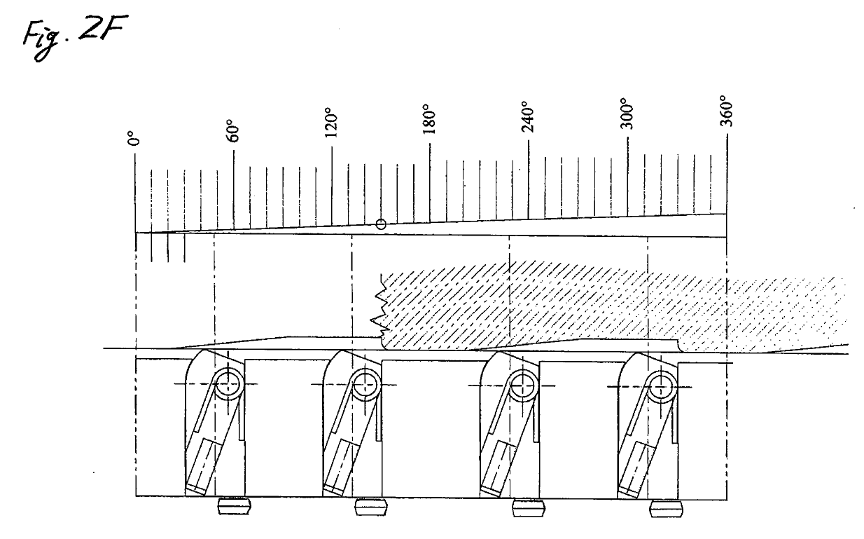

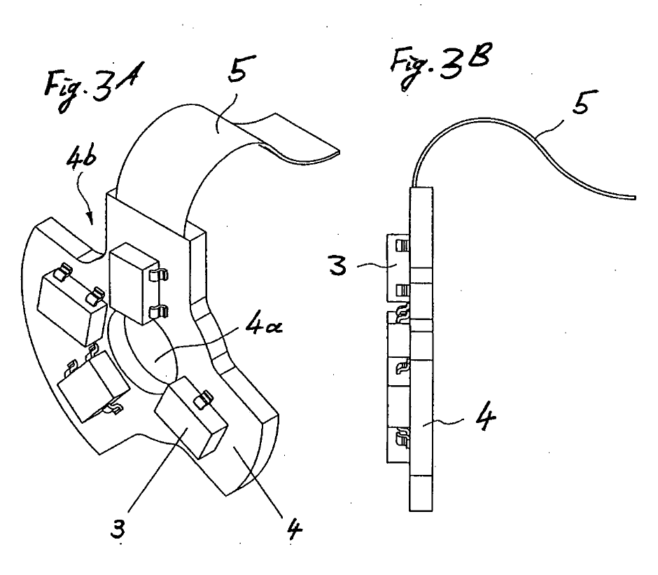

FIGS. 3A and 3B show a perspective view of an embodiment of a circuit board 4 with Hall sensors 3 arranged on it, wherein the circuit board 4 comprises lateral cavities 4b which, in conjunction with the protrusions of the ampoule accommodating device 10 shown in FIG. 1, serve to correctly position the circuit board 4 and therefore the Hall sensors 3 arranged on it. FIG. 3B shows a lateral view of the circuit board 4 shown in FIG. 3A. Other than in the embodiment shown in FIG. 1, the Hall sensors 3 can also be arranged asymmetrically on the circuit board 4, wherein the corresponding transfer elements 7 should advantageously also be arranged asymmetrically on the transfer element support. Other arrangements are possible as well.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An administering device for dispensing a medical substance in doses, comprising a connecting point at which an ampoule can be inserted into the device, wherein in the area of the connecting point, at least one moving transfer element is provided which can either engage with a surface structure of an ampoule or can be moved by a surface structure of the ampoule to transfer surface structure information to a sensor.

2. The administering device according to claim 1, wherein a guide is provided in the area of the connecting point.

3. The administering device according to claim 2, wherein the guide comprises at least one of a groove, a protrusion or a thread.

4. The administering device according to claim 2, wherein the guide comprises a limiting element.

5. The administering device according to claim 1, wherein the at least one transfer element is connected to the device and is moveable relative to the device.

6. The administering device according to claim 5, wherein the at least one transfer element can either be pivoted or shifted relative to the device.

7. The administering device according to claim 1, further comprising a signal-emitting element operably associated with the at least one transfer element.

8. The administering device according to claim 7, wherein the signal-emitting element comprises a permanent magnet.

9. The administering device according to claim 1, wherein the at least one transfer element is biased relative to the device by a biasing element.

10. The administering device according to claim 1, comprising at least two transfer elements.

11. The administering device according to claim 1, wherein the at least one sensor comprises a Hall sensor or a Reed switch.

12. An ampoule for accommodating a medical substance, comprising a surface structure on an ampoule surface, said surface structure comprising at least one of a recess or a protrusion in a circumferential direction.

13. A set of ampoules according to claim 12, wherein different ampoules exhibit different surface structures.

14. A system comprising:
an ampoule for accommodating a medical substance, comprising a surface structure on an ampoule surface, said surface structure comprising at least one of a recess or a protrusion extending in a circumferential direction; and
an administering device for dispensing a medicinal substance in doses, comprising a connecting point at which the ampoule can be inserted into the device, wherein in the area of the connecting point, at least one moving transfer element is provided which can either engage with the surface structure of the ampoule or can be moved by the surface structure of the ampoule to transfer surface structure information to a sensor.

15. An administering device for dispensing a medical substance in doses, comprising a connecting point at which an ampoule can be inserted into the device and at least one moving transfer element adjacent to the connecting point, wherein the at least one moving transfer element can either engage the ampoule or can be moved by the ampoule to transfer ampoule information to a sensor operably associated with the device.
